(19) United States
(12) Patent Application Publication
(10) Pub. No.: US 2015/0305933 A1
(43) Pub. Date: Oct. 29, 2015

(54) INTEGRATED DEVICE SYSTEM AND METHOD FOR NONINVASIVE CORNEAL REFRACTIVE CORRECTIONS

(71) Applicant: Carl Zeiss Meditec AG, Jena (DE)

(72) Inventor: Stephen Q. Zhou, Irvine, CA (US)

(21) Appl. No.: 14/693,982

(22) Filed: Apr. 23, 2015

Related U.S. Application Data
(60) Provisional application No. 61/982,933, filed on Apr. 23, 2014.

(51) Int. Cl. A61F 9/008; A61F 9/009

(52) U.S. Cl. CPC A61F 9/0084; A61F 9/00827; A61F 9/009; A61F 2009/00872; A61F 2009/00895; A61F 2009/00893; A61F 2009/00897

(57) ABSTRACT

A system is provided for noninvasive corneal refractive correction. The system includes an ortho-K lens specifically manufactured based on a topography of a cornea of a human eye for reshaping the cornea from a first configuration to a second configuration. The reshaping can be in situ or as a result of pre-laser treatment. The system also includes a laser device for initiating photochemical crosslinking within an internal layer of the cornea such that the crosslinked cornea remains substantially in the same shape as the second configuration without wearing the ortho-K lens. The laser device includes a laser source configured to produce output radiation in the form of light pulses, a scanner configured to distribute the light pulses in a predetermined pattern, and a light focusing objective configured to focus on an internal space of the cornea and deliver the light pulses into the internal space.

### CROSS REFERENCE TO RELATED APPLICATIONS

This application claims priority to U.S. Provisional Patent Application No. 61/982,933, filed on Apr. 23, 2014, which is hereby incorporated by reference in its entirety.

### FIELD

This invention relates to systems and methods for refractive error corrections for human eyes, and in particular, to systems and methods for myopic correction and other refractive corrections, such as hyperopia, astigmatism, presbyopia, and multifocal vision, as well as for treating keratoconus or post-lasik ectasia.

### BACKGROUND

Laser refractive surgery has been widely accepted as a permanent procedure for correction of myopia, hyperopia, and others refractive errors by ophthalmologists and consumers worldwide. This procedure is irreversible. However laser refractive surgery is an invasive procedure which generally includes (1) creating a hinged flap of corneal surface layer with a microkeratome or laser beams, (2) removing part of the stromal layer by laser light in a predetermined configuration, and (3) replacing the flap back to cover the stromal layer which had been partially removed in a controlled pattern by laser. Because it is an invasive procedure which involves corneal cutting and corneal thinning by removing part of the stromal layer by laser, serious complications can occur. These complications relate to (1) wound healings, such as infection, dry eyes, inflammation, and (2) corneal thinning, such as post-lasik ectasia which may cause visual acuity to worsen relative to the pre-operational visual acuity.

Corneal UV crosslinking is originally developed for keratoconus but recently it is also used for post-lasik ectasia. The procedure includes (1) saturating a human cornea with a solution of riboflavin (Vitamin B₂) solution by instilling drops of riboflavin solution on the cornea surface every 2-5 minutes for about half hour, and (2) exposing the cornea to an UV light for 30-60 minutes with an UV light source of 3 mW/cm². Alternatively, it can also be achieved by exposing the cornea to an extremely high UV light dosage (such as 45 mW/cm²) but short exposure time (such as 5 minutes or less). Riboflavin serves as a photo initiator which produces free radicals upon UV light exposure. These free radicals in turn initiate crosslinkings between collagen molecules of the cornea. By inter-collagen crosslinking, the corneal structure becomes more rigid which freezes the shape of a patient's cornea for an extended period of time or permanently.

On the other hand, temporary and non-invasive procedures for myopia correction also exist. One example is orthokeratology which a patient with mild myopia wears custom-made orthokeratology lens (ortho-K lens) at night time when sleep to temporarily reshape the patient's myopic corneal curvature (first curvature) to a new curvature (second curvature) for emmetropic vision. The next day morning, the patient removes ortho-K lenses and can see without wearing contact lens or eye glasses during day time. Patients must wear the ortho-K lens every night in order to achieve good vision without wearing glasses the next day. If the patient stops wearing ortho-K lens, his or her vision returns to the initial myopic conditions. Recently, there are studies that indicate that ortho-K lenses may slow down or halt myopic regression for young patients. Because myopia is often a progressive disease in young patients, ortho-K has become popular again a decade after FDA approval.

In order to achieve permanent correction for refractive errors with a less invasive procedure, US Patent Application Pub. No. 2009/017305, by Sami G. El Hage in Houston TX, disclosed a combined treatment for corneal crosslinking for a CKR™-treated cornea which results in a long lasting correction of corneal shape and improved vision. CKR stands for controlled kerato-reformation which was used to reshape the corneal shape for patients with refractive errors. The UV crosslinking was achieved by exposing the eye with a UV light of 3 mW/cm² for at least 30 minutes. In separate prior art literature, US Patent Application Pub. No. 2001/0016731 by DeVore and Oefinger, a method was disclosed that accelerates the process for orthokeratology. The method comprises the steps of (1) destabilizing the corneal tissue so that the cornea becomes soft, (2) shaping the softened cornea to the desirable configuration, and (3) restabilizing the softened cornea in the desirable configuration by direct exposure to UV light or visible light in the presence of a photoinitiator. The restabilization method also includes the direct exposure of the cornea to thermal radiation by laser thermal keratoplasty, microwave energy, radio waves, etc. No laser energy delivered directly into an internal layer of the cornea was disclosed. In another prior art patent, U.S. Pat. No. 8,414,911 to Mattson et al, a method for altering mechanical and/or chemical properties of a tissue in a subject (such as human cornea) was described, the method comprising the steps of (a) administering a photoinitiator compound and (b) activating the photoinitiator compound by visible light irradiation of the tissue. Visible light causes insignificant or no damage to the cornea and other eye tissues. Thus, it is an improvement over UV light exposure methods.

On the other hand, the UV light corneal crosslinking suffers from serious damage by the high intensity UV light. Currently commercially available UV crosslinking units carry a UV light source in the range from 3-45 mW/cm². The one used in US Patent Application Pub. No. 2009/017305 has a power of 3 mW/cm², which is considered high and detrimental to the eye tissue. But the procedure requires 30-60 minutes of exposure time for introducing significant corneal crosslinking in the internal layer of a human cornea. To shorten the UV light exposure time, an extremely high dose UV light of 45 mW/cm² has been used for corneal crosslinking with the exposure time in the range of 2-5 minutes. Although riboflavin solution is used to pre-hydrate the cornea for approximately 30 minutes to reduce the risk of UV damage to the eye, the actual damage caused by the direct exposure to the high intensity UV light can be very significant. There are great concerns that the UV light has done too much damage to the eye tissue, especially for a refractive procedure.

Because of the disadvantages of the direct UV exposure technologies described above, US Patent Application Pub. Nos. 2013/03386650 and 2012/0330291 by Jester et al. disclosed laser devices which can be used for corneal crosslinking. Specifically, Jester disclosed that femtosecond lasers with infrared light (700 to 960 nm) can be used to initiate photochemical crosslinking by nonlinear two-photon absorption (or multiple photon absorption) for corneal and other applications. However, such a procedure, when applied to the entire cornea volume treatment, will require as long as 8 hours, which can be impractical for clinical applications.

### SUMMARY OF THE INVENTION

A system is provided for noninvasive corneal refractive correction. The system includes an ortho-K lens specifically manufactured based on a topography of a cornea of a human eye for reshaping the cornea from a first configuration to a second configuration. The reshaping can be in situ or as a result of pre-laser treatment. The system also includes a laser device for initiating photochemical crosslinking within an internal layer of the cornea such that the crosslinked cornea remains substantially in the same shape as the second configuration without wearing the ortho-K lens. The laser device includes a laser source configured to produce output radiation in the form of light pulses, a scanner configured to distribute the light pulses in a predetermined pattern in an x-y plane substantially perpendicular to the optical axis, and a light focusing objective configured to focus on an internal space of the cornea and to deliver the light pulses into the internal space within an internal layer of the cornea.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1A and FIG. 1B depict an eye of a patient fitted with an ortho-K lens configured to reshape the patient's cornea from a first configuration (FIG. 1A) to a second configuration (FIG. 1B);

FIG. 2 depicts elements of a laser device which, in combination with an ortho-K lens pre-treatment, delivers the laser pulses directly into an internal layer of a cornea while reducing surface damage; and

FIG. 3 depicts elements of a laser device which delivers laser pulses directly into an internal layer of a cornea that has an ortho-K lens on its surface (in situ laser treatment).

### DETAILED DESCRIPTION OF PREFERRED EMBODIMENTS

A device and method for permanently correcting refractive errors with a non-invasive procedure could provide great advantages for refractive surgeries. In particular, refractive surgery devices and methods containing a corneal crosslinking device could provide improved functionality over prior art technologies.

One object of an embodiment of the invention is to provide ophthalmologists with an integrated device system which can be used to treat a cornea of mild refractive error with ortho-K lenses for reshaping the patient's cornea from its first configuration to a second configuration in which the patient has emmetropic vision. Once the patient's cornea is stabilized in its second configuration, photo-crosslinking is initiated by a laser device which directly delivers laser light of specific wavelengths into the desired space of the internal layer of the patient's cornea. Alternatively, the patient can be treated with a laser while wearing ortho-K lens in situ. In this alternative method, the cornea is reshaped by the ortho-K lens into the second configuration while the laser treatment locks corneal shape in the second configuration in situ.

For example, when an ultrashort pulse laser is used, the laser pulses initiate the photochemical crosslinking between the collagen molecules in the internal layer of the cornea. Because the laser pulses are focused directly on an internal layer of a human cornea, there is no or minimum damage to the epithelial cells, endothelial cells, and other parts of the eye. On the other hand, in a normal UV light crosslinking scenario, the UV light is focused on the corneal surface, causing serious damage to the epithelial cells and other parts of the eye. In addition, UV light has to penetrate through the corneal surface layer to reach at the internal layer of the cornea wherein the crosslinking occurs. This UV light penetration has reduced effective light intensity, thus leading to a low quantum yield. To compensate the reduced light intensity, the exposure time has to be increased which leads to further damage to the eye. In comparison, laser pulses are focused in the space wherein the crosslinking occurs. Thus, very little light energy is wasted which leads to a high quantum yield and less damage to the eye.

Although corneal crosslinking by laser irradiation with pulses directly targeting an internal layer of the cornea has been disclosed in recent US patent application publications, no prior art literature has been found on the combination of ortho-K lenses with performing a laser corneal crosslinking. For example, US Patent Application Pub. No. 2013/03386650 by Jester, et al at the University of California, Irvine, US Patent Application Pub. No. 2013/0116757 by Russmann at Carl Zeiss Germany, and US Patent Application Pub. No. 2012/0330291 by Jester, et al at the University of California, Irvine disclosed laser devices which can be used for corneal crosslinking. Specifically, Jester disclosed that femtosecond lasers with infrared light (700 to 960 nm) can be used to initiate photochemical crosslinking for corneal and other applications. Jester's application also described utilizing a photoinitiator, such as a riboflavin solution. On the other hand, the Russmann application disclosed a laser device which delivers pulses in the UV range of 260 nm to 290 nm directly into the interlayer of the cornea where the inter-collagen chemical crosslinking occurs. In Russmann's case, it is not necessary to use photoinitiator, such as riboflavin, since these short wavelength UV pulses provide sufficiently high energy to initiate intermolecular crosslinking between collagen chains.

Since neither Jester nor Russmann disclosed the combination device with an ortho-K lens and the method thereof for corneal crosslinking, these prior art references are incorporated into the present invention without further modification.

An object of an embodiment of the invention is to provide ophthalmologists with a new treatment method which comprises (1) obtaining a patient's corneal topography; (2) applying an ortho-K lens, which is made based on the patient's corneal topography, for a short period of time to reshape the patient's cornea from its first configuration to a second configuration wherein the patient achieves emmetropia; and (3) applying laser energy directly targeting the predetermined space of the internal layer of the patient's cornea to perform a crosslinking between collagen molecules of the cornea. As a result, the patient's corneal shape is fixated in its second configuration or in a configuration substantially the same as the second configuration. Consequently, the patient can achieve emmetropia without wearing glasses or ortho-K lenses at night.

An object of an embodiment of the invention is to provide ophthalmologists with an ortho-K lens that can be used to correct refractive errors, such as myopia, hyperopia, and astigmatism on the cornea. After refractive errors are substantially corrected by ortho-K lenses for a short period of time, such as 1-2 weeks, corneal crosslinking can be performed by laser irradiation by the laser system wherein the laser system comprises (1) a laser source, which is structured to produce a radiation output in the form of light pulses; (2) a scanner, which is configured to distribute said light pulses in a receiving area of a microscopic objective; (3) the microscopic objective, which focuses on an internal space of a human cornea and which delivers the incoming light pulses into a space within an internal layer of a patient's cornea according to a predetermined pattern.

An object of an embodiment of the current invention is to provide ophthalmologists with an ortho-K lens which can be used to create multiple focal zones in the cornea with or without combining other surgical procedures. After the desirable corneal surface is established, corneal crosslinking can be performed by laser irradiation by the laser system wherein the laser system comprises (1) a laser source, which is structured to produce a radiation output in the form of light pulses; (2) a scanner, which is configured to distribute said light pulses in a receiving area of a microscopic objective; and (3) the microscopic objective, which focuses on an internal space of a human cornea and which delivers the incoming light pulses into a space within an internal layer of a patient's cornea following a predetermined pattern. The laser pulse scanning pattern will be decided by the specific patient's vision needs and the corneal topography.

An object of an embodiment of the current invention is to provide ophthalmologists with an ortho-K lens and a laser device; the combination of the ortho-K lens and laser treatment allows refractive errors, such as myopia, hyperopia, astigmatism, and presbyopia, to be corrected successfully.

It is an object of an embodiment of the current invention that the laser device and method of use for corneal crosslinking have improved properties in comparison with prior art technologies. Specifically, the laser device of the present invention and its method of use include, for example, an ultrashort pulse laser with a wave length in the UV to visible region which can be directly delivered into the internal space within human corneal stromal layer wherein an ophthalmic dye has been administered prior to the laser treatment. The wavelength of the laser pulse is selected to be approximately equal to or smaller than the absorbance maximum peak (λₘₐₓ) of the ophthalmic dye which serves a photoinitiator. The laser light in this case can enable one-photon absorption of the photoinitiator. Consequently, laser treatment time can be shortened in comparison with Jester's infrared laser pulses for two-photon absorption.

Alternatively, the laser device of the present invention and its method of use can also be a femtosecond laser with long wavelength of approximately 960 nm or higher. Such a laser source of 960 nm or higher is outside of an infrared laser range disclosed by Jester. Laser systems with long wavelength can be used with the combination of ophthalmic dyes or other two-photon initiators which have a peak absorption at approximately 480 nm or higher.

An embodiment of the present invention solves the problem of prior art for the permanent correction of refractive errors which is either an invasive procedure or causing serious damage to the cornea and eye tissue. The problem is solved by providing surgeons with a combination of an ortho-K lens with a laser device. This laser device can be the infrared laser system disclosed in Jester's invention, or preferably improved laser systems which are different from Jester's invention and which overcome deficiencies in Jester's laser system, thus providing superior functionality.

An embodiment of the present invention combines an ortho-K lens, which is used for temporary correction of mild myopia, with corneal crosslinking performed by applying irradiation from a laser system, for example, an improved laser crosslinking system, to create a novel device combination and method of use for permanently correcting refractive errors, such as myopia, hyperopia, and astigmatism. The combined system enables noninvasive procedures which significantly enhance the benefits/risk ratio faced by refractive patients in undergoing the treatment for their refractive correction. Performing corneal crosslinking with direct UV exposure to the human cornea can cause damage to the eye tissue. As a result, refractive patients may not be willing to take the risk of damage for their refractive correction. On the other hand, for patients with progressive keratoconus disease, direct UV light crosslinking, even with high damage to the eye, provides high benefits relative to the keratoconus disease, but not relative to the refractive problems.

The following examples are given for the purpose of illustrating the teachings of the present invention but are not intended to limit the scope of the invention.

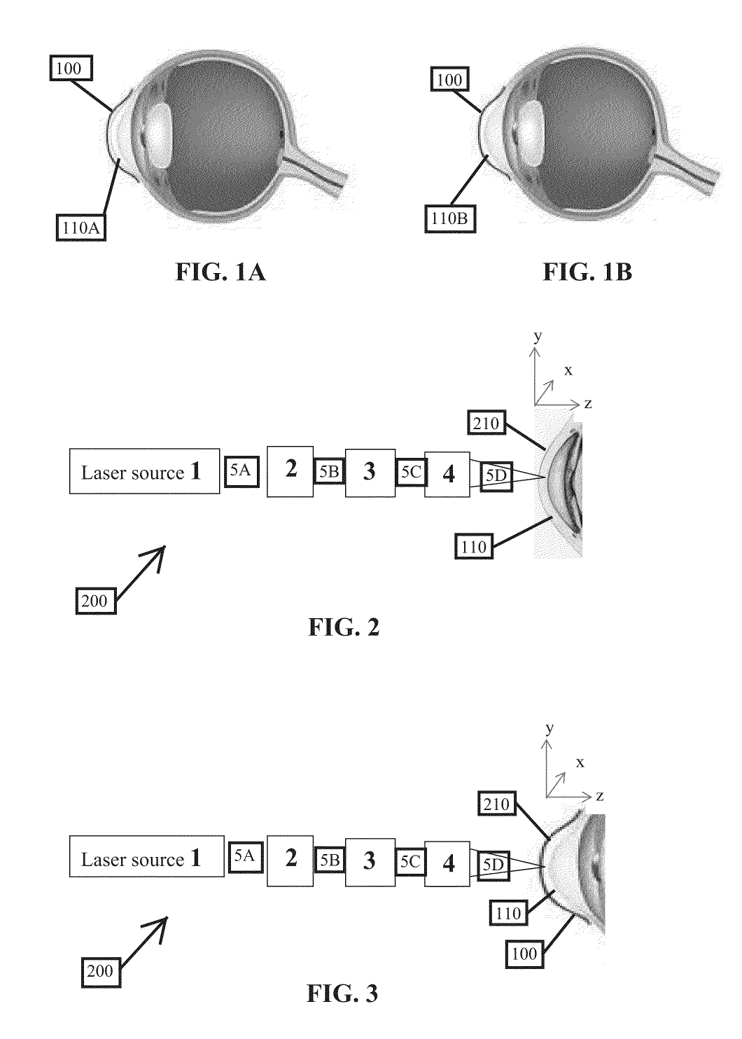

A young patient with progressive myopia is diagnosed with −3.0 D. The patient's corneal topography is taken and a special ortho-K lens is custom-made for the patient. The ortho-K lens 100 exerts more forces in the peripheral region of the cornea 110 to make the myopic cornea 110A of a first configuration more oblate (see FIGS. 1A and 1B). The oblate cornea 110B of a second configuration is depicted in FIG. 1B.

After the patient wears the ortho-K lens for certain period of time, ranging from 2 days to 2 weeks, the patient is refracted again until emmetropia vision is achieved. Once the emmetropia vision is achieved, the patient undergoes a corneal crosslinking procedure. The corneal crosslinking procedure is performed with a laser device and can be performed with the patient wearing the ortho-K lens in situ or without the patient wearing the ortho-K lens.

An embodiment of the present invention for myopic correction includes the following elements: a ortho-K lens for a patient with mild myopia to wear for the purpose of changing the radius of the cornea from a first corneal configuration 110A to a second corneal configuration 110B, and a laser device, such as the laser device 200 depicted in FIGS. 2 and 3.

FIG. 2 depicts elements of a laser device which, in combination with an ortho-K lens pre-treatment, delivers the laser pulses directly into an internal layer of a cornea while reducing surface damage relative to alternative treatments. FIG. 3 depicts elements of a laser device which delivers laser pulses directly into an internal layer of a cornea that has an ortho-K lens 100 on its surface (in situ laser treatment). The laser device 200 includes a laser source 1, a scanner 3, and a focusing objective 4. The laser device 200 also includes a movable lens 2. The movable lens 2 controls the laser pulses in the z-direction along the optical axis and can be positioned in front of the scanner 3, after the scanner 3, inside the objective 4, or some combination thereof. In the embodiments depicted in FIGS. 2 and 3, a photoinitiator 210 is applied to the surface of the cornea 110.

The laser source 1 can be, for example, a femtosecond infrared laser source, a laser source with wavelength in the UV-Visible range (approximately 200 nm-700 nm), or a laser source with long wavelength of approximately 960 nm or higher. Furthermore, in embodiments of the invention, the crosslinking efficiency can be improved by tuning the wavelength of the laser source 1 to be twice a UV absorption peak maximum of the photoinitiator 210. For example, if the photoinitiator 210 is a riboflavin water solution having four absorption peaks at approximately 210 nm, 260 nm, 365 nm, and 450 nm, respectively, a number of options are available for the laser source 1. In order to enhance two photon absorption efficiency (which leads to high quantum yield), the laser source 1 can be an infrared laser source tuned at approximately 730 nm or 900 nm to achieve high efficiency with a riboflavin-treated cornea. Alternatively, the laser source 1 can be a different, non-infrared laser source with a 420 nm or a 520 nm wavelength and achieve high efficiency with a riboflavin-treated cornea. The laser source 1 can also be laser source that provides a combination of laser beams having mixed wavelengths, such as a combination of an infrared laser source with a non-infrared laser source. Such a laser source can be used to treat the cornea for rapid crosslinking. For example, the laser source can be a laser source that provides laser beams with wavelength in the UV-visible range, such as 365 nm or 450 nm to achieve high efficiency with a riboflavin-treated cornea because these wavelengths can cause a combination of one-photon absorption and two-photon absorption, thereby increasing the crosslinking efficiency and consequently reducing laser treatment time.

The laser source 1 can be, e.g., a fixed wavelength fiber laser. The laser source 1 can also be, e.g., a tunable laser such as a ShapeShifter™ (from 200 nm to 10 micron) by Clark-MXR, Inc., or a Mai Tai by Newport, Irvine Calif. The laser source 1 can provide a variable wavelength from 690 nm to 1040 nm with pulse widths as low as 70 femtosecond and source power as high as 5 watts.

This two photon and multi-photon activation for photoinitiators can enable the use of many ophthalmic solutions as the photoinitiator 210, such as, e.g., trypan blue solution. Trypan blue has a peak absorption at approximately 605 nm. This allows one photon absorption by a laser source of 605 nm or two-photon absorption by a femtosecond laser source with a wavelength up to 1210 nm. At this high wavelength, the laser pulse has much better corneal penetration and it causes very little damage to the corneal tissue. Thus, it improves the safety of performing the corneal crosslinking procedure.

Similarly, other ophthalmic dye solutions, such as Fluorescein sodium solution, Rose Bengal, methylene blue, Lissamine green, Indocyanine green, Triamcinolone acetonide, and Brilliant blue solutions can also be used as the photoinitiator 210 for two photon absorption at a wavelength of approximately 960 nm or higher or for one photon absorption at wavelengths in the UV-visible range or for a combination of both one-photon and two-photon absorptions at wavelengths in the UV-visible range by using a femtosecond laser. The advantage of a long wavelength femtosecond laser is that, at the present time, there are many long wavelength femtosecond fiber laser sources commercially available with much higher power than their femtosecond infrared laser counterparts. For example, femtosecond fiber lasers of 1030 nm with a power of 20 watts (pulse energy >100 μj) are available from Amplitude Systemes (France) while no femtosecond fiber lasers with 20 watts of power in the infrared range are commercially available. This high-power, long wavelength femtosecond laser can reduce the treatment time for corneal crosslinking.

More specifically, for one-photon absorption, it is not necessary to have ultrashort laser pulses. Instead the laser source 1 can provide a continuous light beam or ultrashort pulses. Unlike the femtosecond laser disclosed in Jester's application, an embodiment of the invention contemplates the use of a laser source 1 that provides laser beams with a wavelength in the range of UV-visible light (e.g. 200 nm-700 nm) and aims for one photon absorption of the photoinitiator 210, e.g., an ophthalmic dye or other biocompatible photoinitiator, which have been administered on a corneal surface prior to the laser treatment. Therefore, the wavelength of the laser light provided by the laser source 1 is selected to be approximately the same as or smaller than the λₘₐₓ of the photoinitiator 210 administered onto the cornea prior to the laser treatment.

The scanner 3 is a device which delivers laser pulses in the x-y plane which is substantially perpendicular to the optical axis (the z-axis) along which the laser beams produced by the laser source 1 travel from the laser source 1 to the cornea 110. The scanner 3 can be any laser scanner suitable for delivering light pulses into a predetermined pattern in the x-y plane. For example, the scanner 3 can be a set of galvanometric mirrors configured to deflect laser pulses into a predetermined pattern. The scanner 3 can also be a mechanical device, such as an x-y translational stage which moves a fiber laser head together with the objective 4 in a predetermined pattern in the x-y plane. Such a mechanical device can be coupled with an optical device for rapid scanning of laser pulses in the internal layer of the cornea 110.

The objective 4 of the embodiments of the invention depicted in FIGS. 2 and 3 has a clear aperture in a range of approximately 6 mm to 13 mm. It is preferable that the aperture of the objective 4 has a sufficiently large diameter that allows the scanner 3 to move the laser pulses freely over the entire surface of the cornea 110 without refocusing.

The laser pulses in the Z direction along optical axis (z-axis) are controlled by, for example, the movable lens 2. The moveable lens 2 can be positioned after the laser source 1 but in front of the scanner 3, as is depicted in FIGS. 2 and 3. Alternatively, the laser source 1 can be position after the scanner 3 but before the objective, inside the objective 4, or some combination thereof

The actual laser device 200 can be more complicated than the block diagrams depicted in FIGS. 2 and 3 illustrate. In alternative embodiments, a number of additional optical elements can be included in the laser device 200. For example, additional optical elements can be included at any of the positions indicated by blocks 5A, 5B, 5C, and 5D. Such additional optical elements can include, but are not limited to, a beam splitter, a light amplifier, a light modulator, a light detector, a controller (such as a computer), etc. Furthermore, the additional optical elements can include a special device containing a contact lens and placed on the cornea 110 for immobilizing the eye during laser irradiation. As another example, the additional optical elements can include the special ortho-K lens 100 depicted in the embodiment illustrated in FIG. 3 can be placed on cornea 110 for immobilizing the eye and for stabilizing the corneal curvature when corneal crosslinking is performed by laser irradiation.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A system for noninvasive corneal refractive correction, the system comprising:
an ortho-K lens specifically manufactured based on a topography of an individual cornea of a human eye and configured to reshape the individual cornea from a first configuration to a second configuration; and
a laser device for initiating photochemical crosslinking within an internal layer of the individual cornea such that the crosslinked individual cornea remains in substantially the second configuration without the assistance of the ortho-K lens, said laser device comprising:
a laser source configured to produce output radiation in the form of light pulses,
a scanner configured to distribute said light pulses in a predetermined pattern in an x-y plane substantially perpendicular to the optical axis, and
a light focusing objective configured to focus on an internal space of said human cornea and to deliver said light pulses into said internal space within an internal layer of said cornea.

2. The system of claim 1, further comprising a movable lens configured to control the laser pulses in along an optical axis between the laser source and the internal layer of the individual cornea.

3. The system of claim 1, further comprising a photoinitiator applied to the surface of the individual cornea.

4. The system of claim 3, wherein a wavelength of the output radiation produced by the laser source is twice a UV absorption peak maximum of the photoinitiator.

5. The system of claim 1, wherein a wavelength of the output radiation produced by the laser source is in a range of UV-visible light between 200 nm and 700 nm.

6. The system of claim 1, wherein a wavelength of the output radiation produced by the laser source is approximately 960 nm or higher.

7. The system of claim 1, wherein the scanner is a set of galvanometric mirrors configured to deflect laser pulses into a predetermined pattern.

8. The system of claim 1, wherein the scanner is a translational stage configured to move a fiber laser head together with the light-focusing objective in a predetermined pattern in a plane perpendicular to an optical axis along which the laser pulses travel.

9. A method for non-invasive combined corneal refractive correction procedures, the method comprising:
providing an ortho-K lens specifically manufactured based on a topography of an individual cornea of a human eye and configured to reshape the individual cornea from a first configuration to a second configuration; and
delivering laser pulses directly into an internal layer of the individual cornea, the individual cornea being in said second configuration, to perform photochemical crosslinking between collagen molecules in the internal layer of the individual cornea for the purpose of fixating the individual cornea in the second configuration by said laser pulses, the laser pulses being generated by a laser device comprising:
a laser source, said laser source is structured to produce a radiation output in the form of light pulses,
a scanner, said scanner is configured to distribute said light pulses in a predetermined pattern in a x-y plane which is substantially perpendicular to the optical axis, and
a light focusing objective, said objective focuses on an internal space of a human cornea; and said objective delivers the said light pulses into said internal space within an internal layer of said cornea following a predetermined pattern.

10. The method of claim 9, further comprising applying a photoinitiator to the surface of the individual cornea.

11. The method of claim 9, wherein the laser device further comprises a movable lens configured to control the laser pulses in along an optical axis between the laser source and the internal layer of the individual cornea.

12. The method of claim 10, wherein a wavelength of the laser pulses is twice a UV absorption peak maximum of the photoinitiator.

13. The method of claim 9, wherein a wavelength of the laser pulses is in a range of UV-visible light between 200 nm and 700 nm.

14. The method of claim 9, wherein a wavelength of the laser pulses is approximately 960 nm or higher.

15. A system for human corneal crosslinking, the system comprising:
a laser light source configured to output radiation along an optical axis in the form of one of:
a continuous light beam with wavelength in the UV-visible range of approximately 200 nm to 700 nm,
light pulses with wavelength in the UV-visible range of approximately 200 nm to 700 nm, or
light pulses with a wavelength of approximately 960 nm or higher;
a scanner configured to distribute said radiation in a predetermined pattern in an x-y plane substantially perpendicular to the optical axis; and
a light focusing objective configured to focus on an internal space within an internal layer of a human cornea and to deliver the output radiation into the internal space within the human cornea,
wherein the output radiation is absorbed by a photo initiator which is administered onto a surface of the human cornea prior to laser irradiation, and
wherein the output radiation has a wavelength equal to or smaller than the wavelength of the maximum absorption peak (λₘₐₓ) of said initiator.

16. The system of claim 15, further comprising:
an ortho-K lens specifically manufactured based on an individual corneal topography of a human eye for reshaping said individual's cornea from a first configuration to a second configuration as a result of pre-laser treatment.

17. The system of claim 15, further comprising a movable lens configured to control the laser pulses in along an optical axis between the laser source and the internal layer of the individual cornea.

18. The system of claim 15, wherein a wavelength of the output radiation produced by the laser source is twice a UV absorption peak maximum of the photoinitiator.

19. The system of claim 15, wherein the scanner is a set of galvanometric mirrors configured to deflect laser pulses into a predetermined pattern.

20. The system of claim 15, wherein the scanner is a translational stage configured to move a fiber laser head together with the light-focusing objective in a predetermined pattern in a plane perpendicular to an optical axis along which the laser pulses travel.
